(19) United States
(12) Patent Application Publication
(10) Pub. No.: US 2014/0142412 A1
(43) Pub. Date: May 22, 2014

(54) APPARATUS FOR MEASURING DIFFERENCE BETWEEN ANGLES OF PERSON'S NECK

(71) Applicants: Yuji ASAI, Ichinomiya-shi (JP); KS' MOLDING LIMITED COMPANY, Aichi-ken (JP)

(72) Inventor: Yuji ASAI, Ichinomiya-shi (JP)

(73) Assignees: KS' MOLDING LIMITED COMPANY, Aichi-ken (JP); Yuji ASAI, Ichinomiya-shi (JP)

(21) Appl. No.: 13/890,747

(22) Filed: May 9, 2013

(30) Foreign Application Priority Data
Nov. 19, 2012 (JP) 2012-253187

(51) Int. Cl. A61B 5/11; A61B 5/00

(52) U.S. Cl. CPC A61B 5/1121; A61B 5/6814
USPC 600/407

(57) ABSTRACT

An apparatus is provided that measures a difference between angles of a subject's neck in first and second periods of time. The apparatus includes a radiating section fitted to a head of the subject, an image pickup section, which picks up an image of an irradiation plane including an illuminated point based on the radiating section, and a computer. Based on image groups obtained by the image pickup section, the computer computes coordinates of the averages of illuminated points in the first and second periods, respectively. The computer computes the distance between a base point on the plane and each of a pair of perpendicular lines, which each extend orthogonally to a horizontal line included in the plane and further drawn to pass onto the base point, and extend respectively from points of the coordinates of the two averages. The computer computes the neck-angle difference based on the distances.

### CROSS-REFERENCE TO RELATED APPLICATIONS

This application is based upon and claims the benefit of priority from prior Japanese Patent Application No. 2012-253187, filed on Nov. 19, 2012, the entire contents of which are incorporated herein by reference.

### BACKGROUND OF THE INVENTION

The present invention relates to an apparatus for measuring a difference between angles of a person's neck at different times.

It has been evaluated as follows whether or not a person has a disorder in the vestibule of his/her semicircular canal, or about the position sensation of his/her neck.

First, a laser pointer is fitted to a subject's head. Thereafter, the subject is blindfolded or caused to close his/her eyes, and then the subject is caused to direct his/her face precisely to the front. In this state, from the laser pointer, a laser ray is radiated onto a wall or screen (hereinafter referred to as a screen). As illustrated in FIG. 9, an operator gives a mark to a position of a screen 100 (as the above-mentioned screen) where the laser ray is radiated at this time (hereinafter the position will be referred to as a first illuminated point P10). FIGS. 9 and 10 illustrate an example in which the first illuminated point P10 is marked on the screen 100. This first illuminated point P10 is a base point when the subject keeps his/her face forward.

Next, the subject turns his/her neck to direct his/her face either to right or left, and then returns his/her neck to a position at which his/her face is believed to be kept forward. The operator marks a position on the screen 100 where the laser ray is radiated at this time (hereinafter the position will be referred to as a second illuminated point P20).

Thereafter, the operator measures, with a ruler, the distance L10 between the first illuminated point P10 and the second illuminated point P20, each of which is marked on the screen 100.

When the subject's neck is turned, the point which is the center of the rotation of the laser pointer is represented by O.

On the basis of the distance L10 between the two illuminated points, and the distance L20 from the rotation central point O to the screen 100, the operator calculates the angle β between a straight line extending from the rotation central point O to the first illuminated point P1 and a straight line extending from the rotation central point O to the second illuminated point P20 by the triangulation. This angle is used as the difference between the angles of the subject's neck (neck-angle difference).

The evaluation is performed with able-bodied persons, who have no disorder in the respective vestibules of their semicircular canals or about the respective position sensations of their necks. Data about the neck-angle differences obtained in this case are subjected to statistical processing to determine a standard angle range (−γ1<0°<+γ2).

When the angle β is in this standard angle range, the subject has no disorder. When the angle is out of the range, the subject may have a disorder in the vestibule of his/her semicircular canal, or about the position sensation of his/her neck.

In the above described method, the illuminated point obtained when the subject directs his/her neck or face precisely to the front is used as the base point. In another evaluation method, a subject turns his/her neck a plurality of times. Whenever the subject directs his/her face to the front, the angle of his/her neck is measured using, as a base point, the illuminated point obtained in the measurement made immediately before. In this case, a calculation is made to obtain a neck-angle difference between the illuminated point in each of the measurements and the illuminated point in the measurement made immediately before. On the basis of the thus obtained neck-angle differences, it is determined whether or not the subject has a disorder in vestibules of his/her semicircular canals, or about the position sensation of his/her neck.

However, the conventional measuring methods have the following two problems.

The first problem is that any subject's neck is frequently unstable, so that a kinetic fluctuation is generated about each of the first illuminated point P10 and the second illuminated point P20. As a result, the operator, who is marking the screen, arbitrarily selects any one out of a plurality of illuminated points generated by the fluctuation, and then gives the mark thereto. Thus, the marking cannot be precisely attained, so that a large measurement error is caused.

The second problem is as follows. In this measuring method, a measurement is made about a difference between angles of a subject's neck before and after the subject turns his/her neck to right or left. However, in an actual measurement, a shift of his/her neck is unintentionally generated also in the vertical direction between the second illuminated point P20 and the first illuminated point P10, and this unintentional shift is ignored. The neck-angle difference is calculated out from the distance L10 between the first illuminated point P10 and the second illuminated point P20, which includes the shift in the vertical direction. Thus, the measured neck-angle difference includes a component other than components in connection with the right and left directions. FIGS. 9 and 10 illustrate a state in which the second illuminated point P20 is unintentionally shifted upward from the first illuminated point P10.

About the example illustrated in FIGS. 9 and 10, a method for measuring correctly the neck-angle difference along the horizontal direction is made as follows. An intersection point P30 is obtained of a horizontal line Q passing on the first illuminated point P10 and a perpendicular line extending from the second illuminated point P20 to the horizontal line Q. The distance L30 between the intersection point P30 and the first illuminated point P10 is measured. Next, on the basis of this distance L30, and the distance L20, the operator needs to calculate the angle α between a straight line extending from the rotation central point O to the first illuminated point P10, and a straight line extending from the rotation central point O to the intersection point P30 by triangulation.

However, the intersection point P30 is not any actual point illuminated with a ray from the laser pointer. Thus, it is necessary to draw the horizontal line and the perpendicular line on the screen, using a ruler or some other, and then setting up the intersection point P30. As a result, the operation of measuring the distance L30 becomes tiresome. For this reason, the above-mentioned correct measurement method is unpractical. Thus, the simple measurement method has been conventionally made.

Accordingly, the conventional neck-angle difference measuring methods have problems that measurement errors are large and a marking operation is indispensable.

### SUMMARY OF THE INVENTION

Accordingly, it is an objective of the present invention to provide a measuring apparatus capable of measuring correctly a difference between angles of a subject's neck before and after the subject turns his/her neck, without needing to mark any illuminated points.

To achieve the foregoing objective, and in accordance with one aspect of the present invention, an apparatus for measuring a difference between angles of a neck of a subject is provided. The apparatus includes a radiating section, an image pickup section, and a computer. The radiating section is fitted to a head of the subject to radiate a spot ray onto an irradiation plane. The image pickup section picks up an image of the irradiation plane that includes an illuminated point of the spot ray radiated from the radiating section. The image pickup section picks up an image of the irradiation plane in a first period of time for which the subject faces forward, thereby obtaining a first image group including a plurality of images. The image pickup section further picks up an image of the irradiation plane in a second period of time, which is after the neck of the subject either turns horizontally or vertically from the front and subsequently returns the neck of the subject to face forward again, thereby obtaining a second image group comprising a plurality of images. The computer computes a neck-angle difference. The neck-angle difference is defined as a difference between an angle of the neck of the subject in the first period of time and an angle of the neck of the subject in the second period of time. The computer computes coordinates of a first average of illuminated points, each of which corresponds to the illuminated point, on the irradiation plane in the individual images contained in the first image group, and coordinates of a second average of illuminated points, each of which corresponds to the illuminated point, on the irradiation plane in the individual images contained in the second image group, obtains a distance between a base point on the irradiation plane and a first perpendicular line, which extends from a point of the coordinates of the first average and orthogonally to a horizontal line or a vertical line that is included in the irradiation plane and further drawn to pass onto the base point, obtains a distances between the base point and a second perpendicular line, which extends from a point of the coordinates of the second average and orthogonally to the horizontal or the vertical line, and computes the neck-angle difference on the basis of the distance between the first perpendicular line and the base point, the distance between the second perpendicular line and the base point, and the distance between the radiating section and the base point on the irradiation plane.

In accordance with another aspect of the present invention, an apparatus for measuring a difference between angles of a neck of a subject is provided. The apparatus includes a radiating section, an illuminated point detecting section, and a computer. The radiating section is fitted to a head of the subject to radiate a spot ray. The illuminated point detecting section includes an irradiation plane to be irradiated with the spot ray radiated from the radiating section. The illuminated point detecting section detects the position of an illuminated point of the spot ray on the irradiation plane. The illuminated point detecting section detects respective positions of illuminated points, each of which corresponds to the illuminated point, in a first period of time for which the subject faces forward, thereby obtaining a first detection data group of the respective positions of the illuminated points. The illuminated point detecting section further detects respective positions of illuminated points, each of which corresponds to the illuminated point, in a second period of time, which is after the neck of the subject either turns horizontally or vertically from the front and subsequently returns the neck of the subject to face forward again, thereby obtaining a second detection data group of the respective positions of the illuminated points. The computer computes a neck-angle difference. The neck-angle difference defined as a difference between an angle of the neck of the subject in the first period of time and an angle of the neck of the subject in the second period of time. The computer computes coordinates of a first average of the illuminated points on the irradiation plane in the first detection data group, and coordinates of a second average of the illuminated points on the irradiation plane in the second detection data group, obtains a distance between the point of the coordinates of the first average and a perpendicular line that extends from a point of the coordinates of the second average and orthogonally to a horizontal line drawn to pass onto a point of the coordinates of the first average, and computes the neck-angle difference on the basis of the distance between the perpendicular line and the coordinates of the first average, and the distance between the radiating section and the irradiation plane.

Other aspects and advantages of the present invention will become apparent from the following description, taken in conjunction with the accompanying drawings, illustrating by way of example the principles of the invention.

### BRIEF DESCRIPTION OF THE DRAWINGS

The invention, together with objects and advantages thereof, may best be understood by reference to the following description of the presently preferred embodiments together with the accompanying drawings in which:

FIG. 1 is a schematic view of a measuring apparatus according to a first embodiment;

FIG. 2 is a flowchart for computing a difference between angles of a subject's neck before and after the subject turns his/her neck in the first embodiment;

FIG. 3 is an explanatory diagram illustrating a positional relationship among a screen, a radiating section, and an image pickup section;

FIG. 4 is an explanatory diagram illustrating parameters of the image pickup section;

FIG. 5A is an explanatory diagram illustrating a horizontal line H and perpendicular lines V1 and V2;

FIG. 5B is an explanatory diagram illustrating a difference θ between angles of the subject's neck;

FIG. 6 is an explanatory diagram illustrating a vertical line V3 and perpendicular lines H1 and H2 in a modification of the first embodiment;

FIG. 7 is a schematic diagram of a measuring apparatus according to a second embodiment;

FIG. 8 is a flowchart for computing a difference between angles of a subject's neck before and after the subject turns his/her neck in the second embodiment;

FIG. 9 is an explanatory diagram illustrating a difference between angles of a subject's neck before and after the subject turns his/her neck; and

FIG. 10 is an explanatory diagram illustrating distances L10 and L30 on a screen.

### DETAILED DESCRIPTION OF THE PREFERRED EMBODIMENTS

### First Embodiment

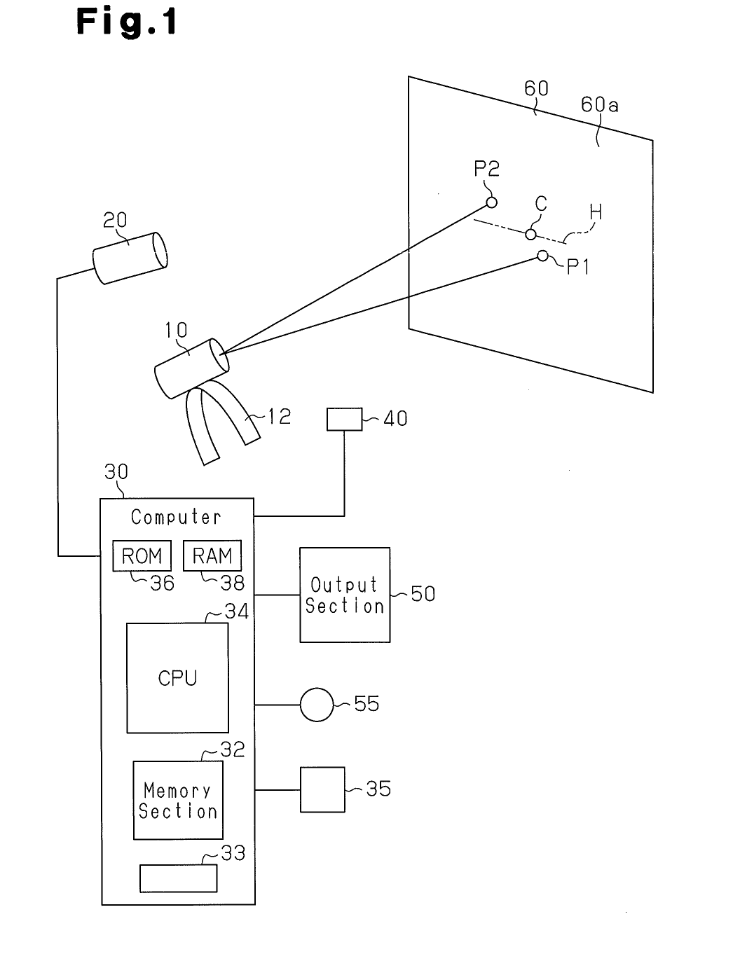

With reference to FIGS. 1 to 4, 5A and 5B, a measuring apparatus according to a first embodiment of the invention will be described.

As illustrated in FIG. 1, the measuring apparatus has a radiating section 10 for radiating a spot ray, an image pickup section 20, a computer 30, a data input section 35, a trigger input section 40, and an output section 50.

The radiating section 10 is attached to a head band 12 fitted to a subject's head to be attachable to his/her head and detachable therefrom. The radiating section 10 is configured to have, for example, a laser pointer that can emit a laser. However, the radiating section 10 is not limited thereto. Thus, the radiating section 10 may be any device as long as it has a light source which can emit a spot ray. When an operator operates an on-off switch (not illustrated), the radiating section 10 emits a light ray or stops the emission. The image pickup section 20 is, for example, a video camera having a solid-state image sensor camera (such as a CCD camera or CMOS camera). In front of the radiating section 10 and the image pickup section 20, a rectangular screen 60 is located which has a flat irradiation plane 60a to be illuminated with light.

The radiating section 10 radiates a spot ray onto the irradiation plane 60a. The image pickup section 20 can pick up an image of the irradiation plane 60a containing a spot where the spot ray is radiated, and pick up a moving image containing a plurality of pictures. The moving image can be obtained, for example, at a rate of 14 frames per second. However, the number of frames per second is not limited to this number.

The image pickup section 20 is connected to a computer 30. The computer 30 has a memory section 32 for memorizing a plurality of images obtained from a moving image captured through the image pickup section 20, a central processing unit (CPU) 34 for processing the images memorized in the memory section 32, a ROM 36, a RAM 38, and a clocking timer 33. The ROM 36 memorizes control programs for controlling the whole of the system of the computer 30, and a neck-angle difference calculating program. The RAM 38 is a working memory used when the CPU 34 conducts any processing.

The computer 30 is connected to the data input section 35, the trigger input section 40, and a sound generating section 55 for generating a warning message, such as a buzzer or a speaker. The data input section 35 is, for example, a keyboard. Through the data input section 35, parameters used for various image processings are input to the CPU. The trigger input section 40 has a trigger button (not illustrated), which is operated by any subject and any operator. When the trigger button is operated to be switched on, a trigger signal is output to the computer 30. The computer 30 is also connected to the output section 50. The computer 30 can output various calculated results to the output section 50. The output section 50 is, for example, a display and a printer.

### (Operation of First Embodiment)

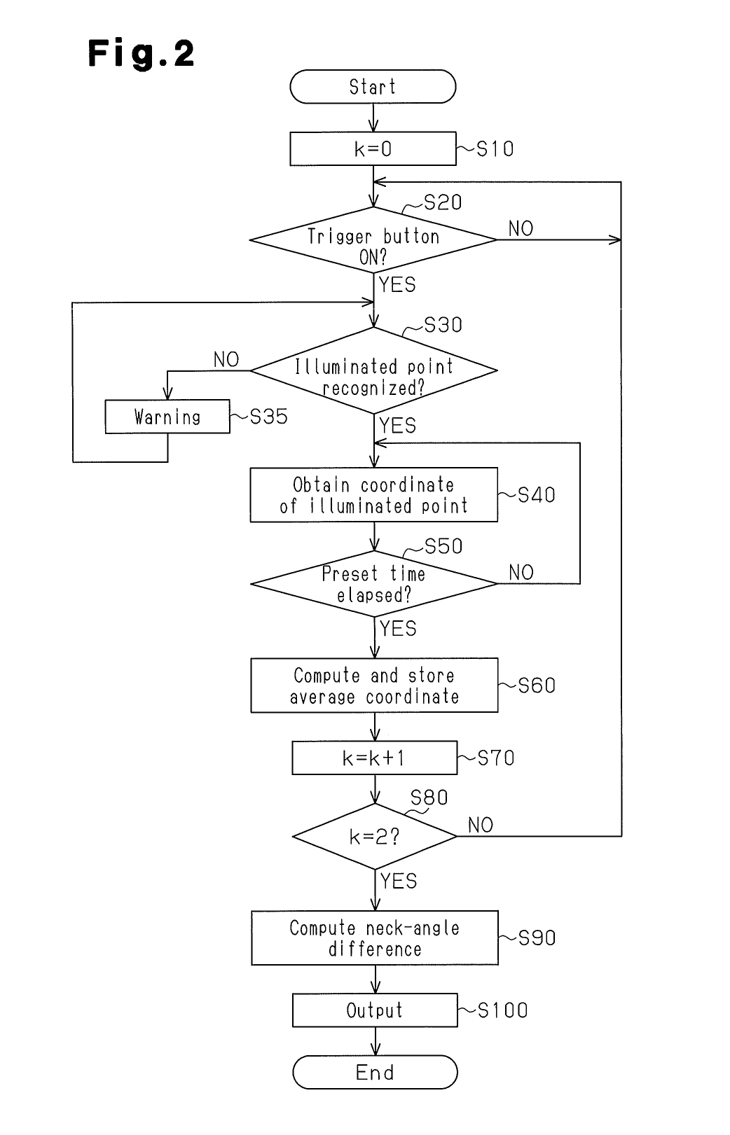

With reference to FIGS. 2 to 4, a description will be made about operation of the measuring apparatus according to the first embodiment.

### (Preparation Stage)

Before an operator measures the angle of a subject's neck, the operator first inputs, through the data input section 35, parameters necessary for setting up a viewing angle of the image pickup section 20.

The parameters to be input are obtained as follows. The image pickup section 20 is arranged at a position apart from the screen 60 by a camera distance D2 for measurement, or by a distance D3 shorter than the measurement camera distance D2. The height and the direction of the image pickup section 20 are set to cause the optical axis of lenses of the image pickup section 20 to cross the irradiation plane 60a of the screen 60 orthogonally at a center C of the irradiation plane 60a. In order to reduce measurement errors, it is preferred to set the image pickup section 20 at a position apart from the screen 60 by the measurement camera distance D2. However, the position where the image pickup section 20 is set is not limited to this position. The height and the direction of the image pickup section 20 are set, using the center C as a base point. However, the center C is a mere example. A different position may be used as the base point.

In this state, the operator measures the size of an image pickup region 60b of the irradiation plane 60a of the screen 60 that can be picked up through the image pickup section 20. Specifically, the operator picks up an image of the irradiation plane 60a through the image pickup section 20. On the basis of the image picked up through the image pickup section 20, in the irradiation plane 60a of the screen 60, a mark is given to a boundary between the image pickup region 60b and a region the image has not been picked up. Next, the height h1 and the width w1 of the rectangular image pickup region 60h are measured with a ruler.

FIG. 4 shows the height h1 and the width w1 of the rectangular image pickup region 60b obtained when the image pickup section 20 at this preparation stage is placed onto the position apart from the screen 60 by the distance D3, which is shorter than the measurement camera distance D2. The distance D3 may be, for example, 1 m. However, the distance D3 is not limited to this distance. FIG. 4 shows the height h and the width w of the rectangular image pickup region 60b obtained when the distance between the image pickup section 20 and the screen 60 at this preparation stage is equal to the measurement camera distance D2.

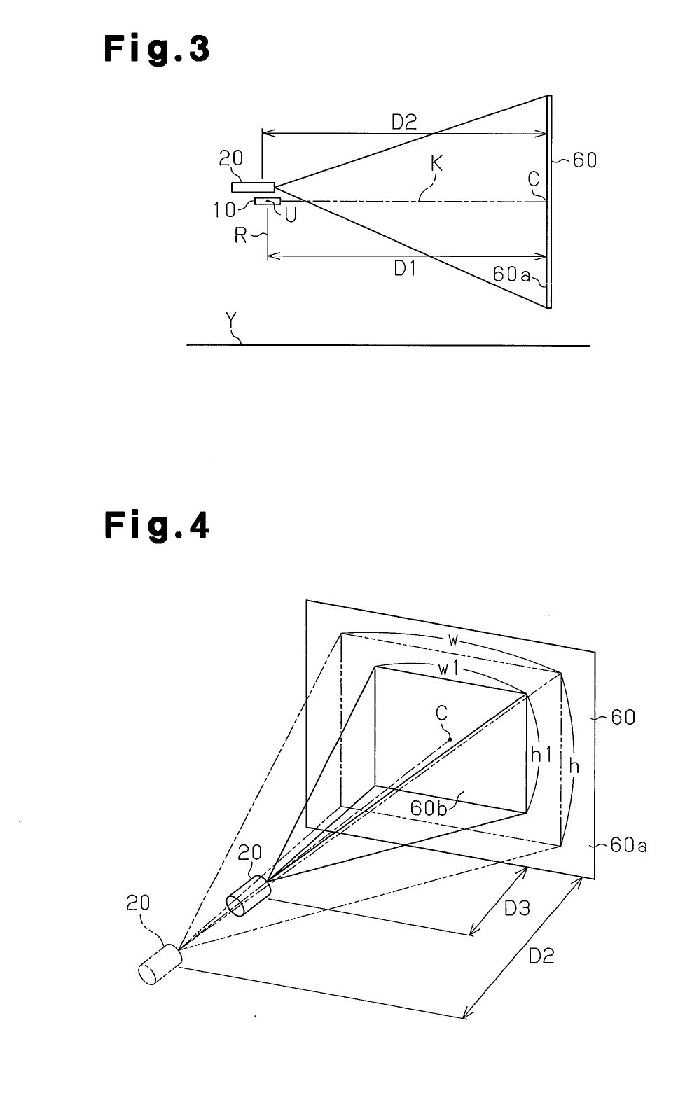

Next, as illustrated in FIG. 3, the operator causes a subject, the radiating section 10 being fitted to his/her head using a head band 12, to be positioned such that an optical axis K of the radiating section 10 crosses the irradiation plane 60a orthogonally at the center C of the irradiation plane 60a of the screen 60. At this time, the operator measures the distance D1 between the radiating section 10 and the screen 60 with a ruler. As illustrated in FIG. 3, the distance D1 is the distance from an intersection point U to the center C of the irradiation plane 60a. The point U is an intersection point of a center line R of the rotation of the subject's head when the subject turns his/her neck horizontally, and the optical axis of the radiating section 10. The distance D1 between the radiating section 10 and the screen 60 is measured such that the subject's head is not shifted unintentionally, that is, in the state in which his/her head is stable. At this time, it is unnecessary to emit a spot ray from the radiating section 10. In the present description, any wording such as the wording “neck turns horizontally” has the same meaning as any wording such as the wording “head turns horizontally”.

The operator arranges the image pickup section 20 to make the center C of the irradiation plane 60a of the screen 60 consistent with the center of an image picked up through the image pickup section 20 and further not to project the radiating section 10 and the subject into the picked-up image. At this time, the height of the optical axis of the lenses of the image pickup section 20 from the floor surface is set to be equal to that of the optical axis K of the radiating section 10. The image pickup section 20 is arranged to direct the optical axis of the lenses of the image pickup section 20 into the center C of the irradiation plane 60a of the screen 60 and further make this optical axis near the optical axis K of the radiating section 10. When the image pickup section 20 is arranged to make the optical axis of the lenses of the image pickup section 20 near the optical axis K of the radiating section 10 in this way, an accidental error is minimized in the calculation of the neck-angle difference. As the distance D1 and the distance D2 are each longer, this error can be made smaller. Thus, the error can be favorably ignored.

In FIG. 3, the image pickup section 20 is illustrated to be shifted upward from the radiating section 10 for illustrative purposes. It is preferred to make the optical axis K of the radiating section 10 concentric with that of the lenses of the image pickup section 20. However, since the radiating section 10 is fitted to the subject; it is difficult to make the two optical axes concentric with each other physically. Thus, the image pickup section 20 is arranged to be shifted as described above.

At this time, the operator measures the distance D2 between the image pickup section 20 and the screen 60 (camera distance) with a ruler. The heights h1 and h, and the widths w1 and w of the image pickup region 60b, and the distances D1 and D2 are parameters obtained at this preparation stage.

The operator uses the data input section 35 to input these parameters into the computer 30, and the CPU 34 stores the parameters into the memory section 32. Furthermore, the operator uses the data input section 35 to input a preset time t determined to specify the length of a time for a moving image to be processed. The CPU 34 stores the preset time t into the memory section 32. The preset time t has a unit of ms. The preset time t is preferably, for example, from 100 to 10000 ms. However, the time t is not limited thereto. The preset time t may be appropriately set by the operator in accordance with the environment where the screen is to be imaged, for example, the processing capacity of the used computer 30, or the environment of lighting for the place for the measurement, and may be input through the data input section 35.

### (Measurement of Difference Between Angles of Subject's Neck)

The subject puts the radiating section 10 on his/her head using the head band 12, and directs his/her face to the front in the state of being blindfolded or closing his/her eyes. At this time, in accordance with instructions from, for example, the operator, who is in attendance on the subject, the subject directs his/her face to the front to hit a spot ray to be emitted from the subject onto or near a point given beforehand to the irradiation plane 60a. In this state, from the radiating section 10, a spot ray is radiated toward the irradiation plane 60a of the screen 60, and the image pickup section 20 starts to pick up a moving image of the screen.

When the neck-angle difference calculating program is started, the CPU 34 carries out individual steps of a flowchart shown in FIG. 2.

### (Step S10)

In step S10, the CPU 34 sets the value k of the counter to 0.

### (Step S20)

In step S20, the CPU 34 awaits an input of a trigger signal from the trigger input section 40. The subject or the operator operates the trigger input section 40 to input the trigger signal to the CPU 34. The CPU 34 then proceeds to step S30.

### (Step S30)

In step S30, the CPU 34 obtains a picked-up image included in the moving image picked-up through the image pickup section 20 in the unit of each of its frames. Each of the picked-up images is subjected to a predetermined digital processing. Each of the picked-up images is gradated in accordance with predetermined luminance gradations (for example, a gray scale of 256 gradations), or binarized by use of a predetermined luminance as a threshold value. The processed image is referred to as the “digital image”. Next, it is determined whether or not the CPU 34 has recognized an illuminated point of the spot ray in the digital image. A region having the illuminated point is higher in luminance than any region having no illuminated point. Thus, when the digital image, which is gradated in accordance with the luminance gradations, has a region having a higher luminance than an appropriately-set reference luminance, the CPU 34 recognizes the region to be the illuminated point. In the case of the binarized image, the threshold value is set, for example, such that only the illuminated point is to be a white region. When the CPU 34 identifies the presence of this white region, the CPU 34 recognizes the region to be the illuminated point.

The CPU 34 causes the output section 50, such as a display, to display the picked-up image, and a symbol surrounding the recognized illuminated point in the state in which the symbol overlaps with the image. At this time, in the output section 50, the symbol is displayed on the picked-up image, which is an actual image. The illuminated point on the actual image is surrounded by the symbol. In this case, the shape of the symbol is not limited, and is, for example, a square frame.

When the CPU 34 cannot recognize the illuminated point, which is illuminated with the spot ray, in step S30, the CPU 34 proceeds to step S35 to issue a warning, and then returns to step S30 to wait until the CPU 34 can recognize the spot-ray illuminated point. The warning is issued, for example, by producing a buzzer sound from the sound generating section 55, or giving a warning message that no spot-ray illuminated point can be recognized according to synthetic sounds.

When the CPU 34 has recognized the spot-ray illuminated point in step S30, the CPU 34 starts to measure time through the timer 33, and proceeds to step S40. Specifically, the CPU 34 proceeds to step S40 when the trigger signal is input in step S20 and further the spot-ray illuminated point is recognized in step S30.

### (Step S40)

In step S40, the CPU 34 computes the coordinates of the illuminated point recognized in the digital image of each of the frames obtained while the timer 33 measures time. When the recognized illuminated point is made not of only one pixel but of a plurality of pixels, the coordinates of a pixel present at the center of regions composed of the pixels are computed.

The digital image is rectangular, and is composed of m×n pixels wherein m is the number of pixels in each of rows in the horizontal direction, and n is that of pixels in each of columns in the vertical direction. Thus, about the coordinates (x, y) of the recognized illuminated point, any point in the digital image may be decided as the origin. For example, the center of the digital image may be decided as the origin of the digital image coordinate system. However, the origin is not limited to the center. The above-mentioned picked-up images are each picked up such that the center of the digital image is consistent with the center C of the irradiation plane 60a.

The time when the value k of the counter is 0 is a time when the subject firstly keeps his/her face forward. Digital images obtained from a moving image picked up when the subject first keeps his/her face forward are referred to as a first image group. A period of time from the time when the timer 33 starts to measure time in the first neck-angle measurement to a time when the preset time t elapses is referred to as a first period of time.

The CPU 34 obtains the coordinates (x, y) of the recognized illuminated point on the digital image, and then converts the coordinates to coordinates (X, Y) on the irradiation plane 60a. In other words, the coordinates of the recognized illuminated point in the digital image coordinate system are converted to coordinates of the illuminated point in the screen coordinate system. When the coordinate conversion is finished, the CPU 34 proceeds to step S50.

The coordinate conversion will now be described herein.

The coordinates (x, y) are converted as follows in the case of placing, in the preparation stage as illustrated in FIG. 4, the image pickup section 20 apart from the screen 60 by the distance D3 and measuring the height h1 and the width w1 of the image pickup region 60b.

The X coordinate of the illuminated point in the screen coordinate system is obtained by multiplying the coordinate x of the illuminated point in the digital image coordinate system by a conversion factor D2×w1/(D3×m).

The Y coordinate of the illuminated point in the screen coordinate system is obtained by multiplying the coordinate y of the illuminated point in the digital image coordinate system by a conversion factor D2×h1/(D3×n).

The coordinates (x, y) are converted as follows in the case of placing, in the preparation stage as illustrated in FIG. 4, the image pickup section 20 apart from the screen 60 by the measurement camera distance D2, and measuring the height h and the width w of the image pickup region.

The X coordinate of the illuminated point in the screen coordinate system is obtained by multiplying the coordinate x of the illuminated point in the digital image coordinate system by a conversion factor w/m. The Y coordinate of the illuminated point in the screen coordinate system is obtained by multiplying the coordinate y of the illuminated point in the digital image coordinate system by a conversion factor h/n.

### (Step S50)

In step S50, the CPU 34 memorizes, in the memory section 32, the coordinates of the illuminated point in the screen coordinate system, and then compares the time measured by the timer 33 with the preset time t. When the time measured by the timer 33 has not elapsed by the preset time t, the CPU 34 returns to step S40, and then converts the coordinates of the recognized illuminated point in the digital image coordinate system to coordinates thereof in the screen coordinate system, as described above. Accordingly, coordinates of a plurality of illuminated points are memorized until the time (measured by the timer 33) elapses by the preset time t. When the time measured by the timer 33 has elapsed by the preset time t, the CPU 34 proceeds to step S60.

### (Step S60)

In step S60, the CPU 34 averages the respective coordinates of the illuminated points, in the screen coordinate system, obtained until the time elapses by the preset time t, to compute the coordinates of the average of the illuminated points. The coordinates of the average are memorized in the memory section 32, correspondingly to the value k of the counter.

When the value k of the counter is, for example, 0, the resultant coordinates of the average are memorized in the memory section 32 as the coordinates of the average of the illuminated points when the value k of the counter is 0. In the first embodiment, the coordinates of the average of the illuminated points when the value k of the counter is 0 are referred to as coordinates of a first average.

When the CPU 34 has obtained the coordinates of the average of the illuminated points, the CPU 34 causes the output section 50, for example, a display screen of a display, to display a symbol showing the position of the coordinates of the average. The symbol showing the position of the coordinates of the average is displayed in a form or color different from that of the symbol showing the above-mentioned recognized illuminated point. In the first embodiment, the symbol showing the position of the coordinates of the average is a circular frame. However, the symbol is not limited thereto.

### (Step S70)

When step S60 is finished, the CPU 34 proceeds to step S70. In step S70, the CPU 34 increases the value k of the counter by one.

### (Step S80)

When the step S70 is finished, the CPU 34 determines whether or not the value k of the counter is 2 in step S80. When the value k of the counter is not 2, the CPU 34 returns to step S20.

### (Step S20 to Step S70 in Secondarily Performed Process)

When the program is returned from step S80 to step S20, the subject turns his/her neck to either right or left from the state in which the subject keeps his/her face forward. Thereafter, the subject returns his/her neck to a position at which his/her face is believed to be kept forward. Thereafter, the subject operates the trigger input section 40. Alternatively, after the subject returns his/her neck, the operator operates the trigger input section 40. In this way, the CPU 34 proceeds from step S20 to step S30, and performs the processing up to step S60 in the same manner as performed in the first neck-angle measurement. In step S60 in the second neck-angle measurement, the CPU 34 computes the coordinates of the average of the illuminated points in the screen coordinate system when the value k of the counter is 1, and then causes the coordinates of the average to be memorized into the memory section 32 to correspond to the value k of the counter. The coordinates of the average when the value k of the counter is 1 are referred to as coordinates of a second average.

Digital images obtained in step S40 from a moving image picked up when the subject keeps his/her face forward in the second neck-angle measurement are referred to as a second image group. A period of time from the time when time starts to be measured by the timer 33 in the second neck-angle measurement to a time when the preset time t elapses is referred to as a second period of time.

In step S70, the CPU 34 increases the value k of the counter by one. Specifically, in step S70 in the second neck-angle measurement, the CPU 34 sets the value k of the counter to 2, and proceeds to step S80.

In step S80 in the second neck-angle measurement, the value k of the counter is 2, so that the CPU 34 proceeds to step S90.

### (Step S90)

In step S90, the CPU 34 computes the neck-angle difference.

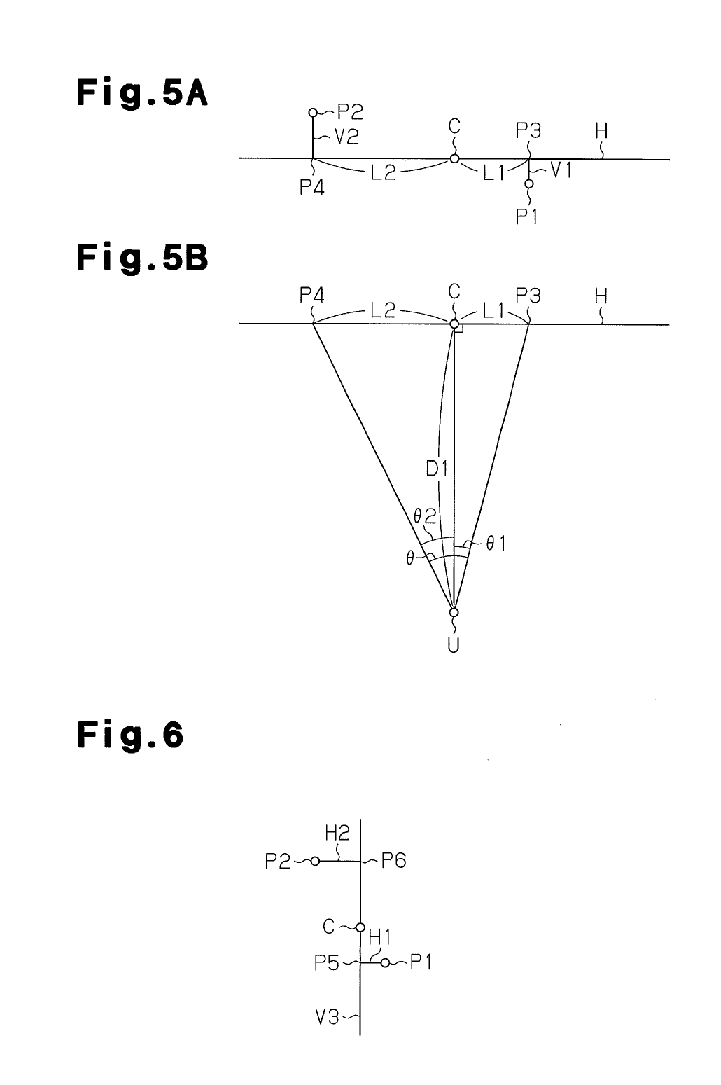

With reference to FIGS. 1, 5A and 5B, a method for computing the neck-angle difference will be described. The illuminated point of coordinates of the first average on the screen 60 is represented by P1; and the illuminated point of coordinates of the second average thereon is represented by P2.

As illustrated in FIG. 5A, the following intersection point is set as P3 on the irradiation plane 60a: an intersection point of a horizontal line H drawn to pass onto the center C and a first perpendicular line V1 extending perpendicularly to the horizontal line H from the spot-ray illuminated point P1 in the first period of time to the horizontal line H. The following intersection point is set as P4 on the irradiation plane 60a: an intersection point of the horizontal line H and a second perpendicular line V2 extending perpendicularly to the horizontal line H from the spot-ray illuminated point P2 in the second period of time to the horizontal line H. The CPU 34 computes, on the screen coordinate system, coordinates of the intersection point P3 and those of the intersection point P4 from the coordinates of the first average of the illuminated point P1 and the coordinates of the second average of the illuminated point P2. Next, the CPU 34 uses the respective coordinates of the intersection points P3 and P4, and the coordinates of the center C of the irradiation plane 60a to compute the distance L1 from the intersection point P3 of the horizontal line H and the first perpendicular line V1 to the center C, and the distance L2 from the intersection point P4 of the horizontal line H and the second perpendicular line V2 to the center C. The distance L1 is the distance from the center C to the first perpendicular line V1, and the distance L2 is the distance from the center C to the second perpendicular line V2.

The CPU 34 uses the distances D1 and L1 to compute the angle θ1 between a straight line extending from the intersection point U shown in FIG. 5B to the center C of the irradiation plane 60a and a straight line extending from the intersection point U to the intersection point P3 by the triangulation. The CPU 34 also uses the distances D1 and L2 to compute the angle θ2 between the straight line extending from the intersection point U to the center C of the irradiation plane 60a, and a straight line extending from the intersection point U to the intersection point P4 by the triangulation. The CPU 34 adds θ1 and θ2 to each other to compute the neck-angle difference θ.

### (Step S100)

In step S100, the CPU 34 causes the neck-angle difference θ to be displayed on the output section 50, such as the display and the printer. The CPU 34 then ends the present neck-angle difference calculating program.

The first embodiment has the following characteristics.

(1) The measuring apparatus of the first embodiment has the radiating section 10 fitted to a subject's head to radiate a spot ray onto an irradiation plane and the image pickup section 20, which picks up images of the irradiation plane 60a including an illuminated point of the spot ray radiated from the radiating section. The image pickup section 20 picks up images of the irradiation plane in the first period of time, while the subject keeps his/her face forward, thereby obtaining a first image group including a plurality of images, and picks up images of the irradiation plane in the second period of time, which is after the subject turns his/her neck horizontally from the front and subsequently returns his/her neck or head to keep his/her face forward again, thereby obtaining a second image group including a plurality of images. The measuring apparatus further has the computer 30, which computes a difference between the angle of the subject's neck in the first period of time, and that in the second period of time. The computer 30 computes coordinates of the first average of illuminated points, each of which corresponds to the illuminated point, on the irradiation plane 60a in the individual images contained in the first image group, and coordinates of the second average of illuminated points, each of which corresponds to the illuminated point, on the irradiation plane 60a in the individual images contained in the second image group. Furthermore, the computer 30 obtains the distance between the center C (base point) on the irradiation plane 60a and the first perpendicular line V1, which extends from the coordinates of the first average and orthogonally to the horizontal line H included in the irradiation plane 60a and is further drawn to pass onto the center C. The computer 30 also obtains the distance between the center C and the second perpendicular line V2, which extends from the coordinates of the second average and orthogonally to the horizontal line H and is further drawn to pass onto the center C. Next, the computer 30 computes the neck-angle difference θ on the basis of the distance between the first perpendicular line V1 and the center C (base point), the distance between the second perpendicular line V2 and the center C (base point), and the distance between the radiating section 10 and the center C (base point) on the irradiation plane 60a.

As a result, according to the measuring apparatus of the first embodiment, the neck-angle difference can be precisely measured without needing to give a mark onto any one of the illuminated points.

(2) The measuring apparatus of the first embodiment has the trigger input section 40 for inputting a trigger signal into the computer 30. When the trigger signal is input from the trigger input section 40 to the computer 30, the timer 33 is activated to start measuring the first and second periods of time. When the time measured by the timer 33 has elapsed by the preset time t, the first and second periods of time each end.

According to the measuring apparatus of the first embodiment, after an operating person who operates the trigger input section 40 has operated the trigger input section 40, any image through the image pickup section 20 can be obtained. Thus, when the operating person is the subject, any picked-up image can be obtained whenever the subject desires to pick up the image. In other words, some time may be required for measuring-preparation according to the subject; thus, any image can be obtained in accordance with subject's will. When the operating person is the operator, the operator operates the trigger input section at a stage when measuring-preparation is finished according to the subject, whereby any image can be obtained in accordance with subject's will.

### Second Embodiment

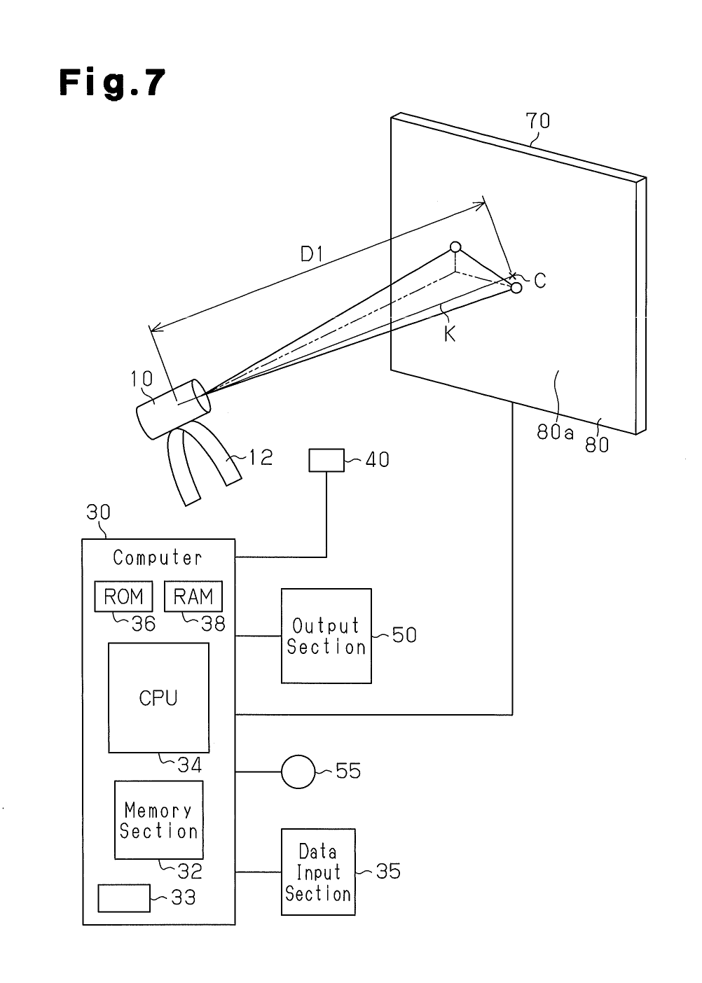

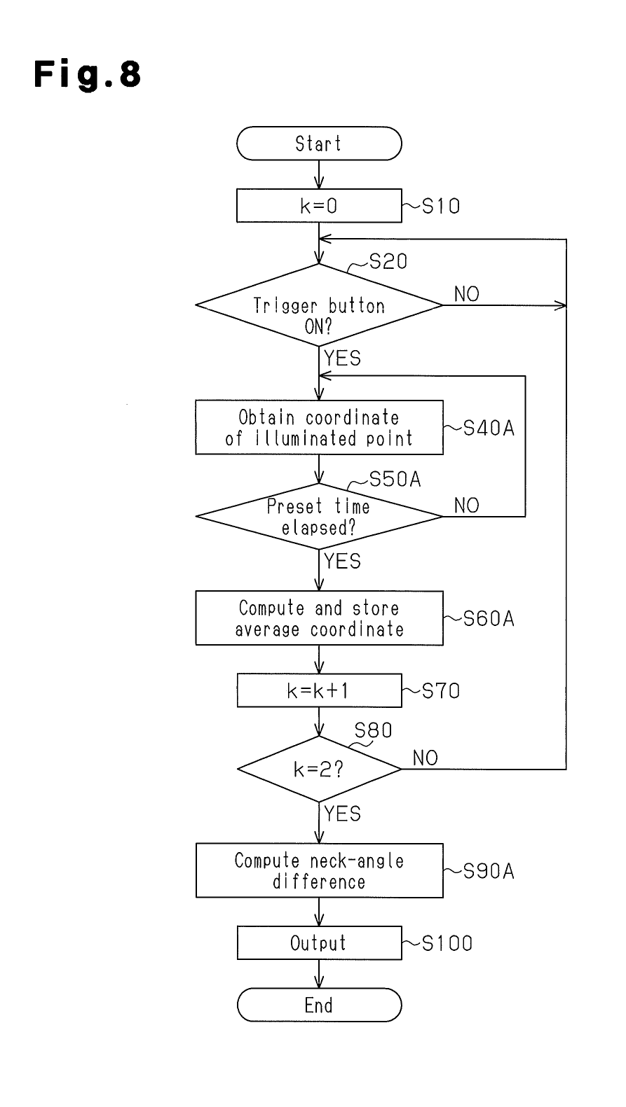

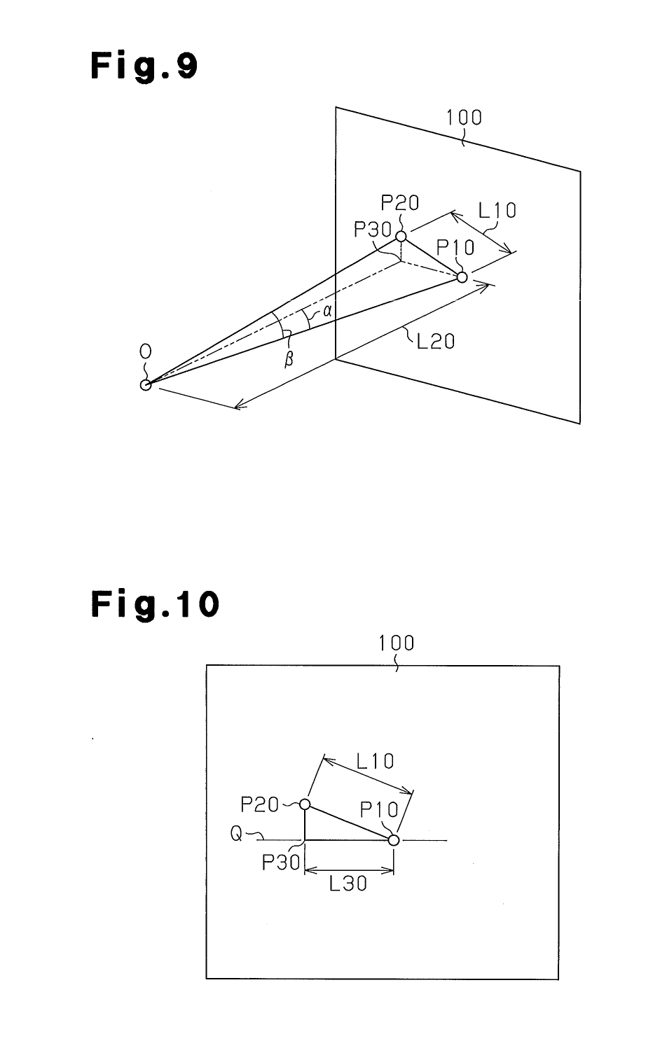

With reference to FIGS. 7 and 8, a description will be made about a measuring apparatus according to a second embodiment. About the second embodiment, the description is directed mainly to components different from those of the measuring apparatus of the first embodiment. The same reference numbers or symbols as in the first embodiment are attached to the components of the second embodiment that are equivalent to or correspond to those of the measuring apparatus of the first embodiment, in which the subject has turned his/her neck horizontally. Description thereof is not repeated here.

The measuring apparatus of the second embodiment is different in structure from the first embodiment in that the image pickup section 20 is omitted. Further, instead of the screen 60, an illuminated point detecting section 70 is provided that has a spot ray detecting plate 80. The spot ray detecting plate 80 is equipped with a semitransparent plate having a flat irradiation plane 80a, and a photodiode array located on the back of the semitransparent plate. The array has a plurality of photodiodes arranged in the XY directions. The photodiode array is connected to the computer 30. In the photodiode array, the position (i.e., the XY coordinates) of a photodiode that has detected a spot ray is identified by the computer 30.

### (Operation of Second Embodiment)

With reference to FIG. 8, operation of the measuring apparatus according to the second embodiment will be described.

### (Preparation Stage)

As illustrated in FIG. 8, before an operator measures the angle of a subject's neck, the radiating section 10 is first fitted to the subject's head using the head band 12. The operator causes the subject to be positioned to make the optical axis K of the radiating section 10 perpendicular to the irradiation plane 80a of the illuminated point detecting section 70 at the center C of the irradiation plane 80a. At this time, the operator measures the distance D1 between the radiating section 10 and the irradiation plane 80a with a ruler in the same way as in the first embodiment.

The distance D1 is a parameter obtained at this preparation stage. The operator arranges the radiating section 10 to make the optical axis K of the radiating section 10 perpendicular or substantially perpendicular to the irradiation plane 80a of the illuminated point detecting section 70 at the center C of the irradiation plane 80a. The center C of the irradiation plane 80a corresponds to a base point. The operator uses the data input section 35 to input the parameter into the computer 30, and the CPU 34 stores the parameter into the memory section 32. Furthermore, in the same way as in the first embodiment, the operator uses the data input section 35 to input a preset time t determined to specify the length of a time for a moving image to be processed. The CPU 34 stores the preset time t into the memory section 32.

### (Measurement of Difference Between Angles of Subject's Neck)

In the same manner as in the first embodiment, the subject puts the radiating section 10 on his/her head using the head band 12, and directs his/her face to the front in the state of being blindfolded or closing his/her eyes while a spot ray is emitted from the radiating section 10.

When the neck-angle difference calculating program is started, the CPU 34 carries out individual steps of a flowchart shown in FIG. 8. The flowchart in FIG. 8 is different from that of the first embodiment shown in FIG. 2 in that steps S30 and S35 are omitted and further steps S40A to step S60A, and step S90A are performed instead of steps S40 to S60, and step S90.

Hereinafter, the steps different from those in the first embodiment will be described, and description of the same steps as in the first embodiment is not repeated. In the second embodiment, a trigger button in the trigger input section 40 is switched on in step S20. As a result thereof, the timer 33 starts to measure time.

### (Step S40A)

In step S40A, the CPU 34 obtains whether or not the photodiode array has detected the spot ray in each detecting cycle while time is measured by the timer 33 until the preset time t elapses. The detecting cycle is a shorter time period than the preset time t. When the photodiodes of the photodiode array include a photodiode that has detected the spot ray, the CPU 34 identifies the coordinates of the photodiode from a non-illustrated table stored in the memory section 32. In other words, the CPU 34 obtains the coordinates (X, Y) of the illuminated point in the screen coordinate system. When a plurality of photodiodes of all the photodiodes have detected the spot ray, the position of the photodiode near the center of the illuminated point is determined as the coordinates (X, Y) of the illuminated point in the screen coordinate system.

The time when the value k of the counter is 0 is a time when the subject first keeps his/her face forward. The following group is referred to as a first detection data group: a group of position detection data that includes data on respective positions where, in this case, the spot ray is detected in the individual detecting cycles of the photodiode array. A period of time from the time when the timer 33 starts to measure time in the first neck-angle measurement to a time when the preset time t elapses is referred to as a first period of time.

The following group is referred to as a second detection data group: a group of position detection data that are data on respective positions where the spot ray is detected in the individual detecting cycles of the photodiode array when the subject keeps his/her face forward in a second neck-angle measurement in step S40A. A period of time from the time when the timer 33 measures time in the second neck-angle measurement to a time when the preset time t elapses is referred to as a second period of time.

### (Step S50A)

In step S50A, the CPU 34 memorizes, in the memory section 32, the coordinates of the illuminated point, and then compares the time measured by the timer 33 with the preset time t. When the time measured by the timer 33 has not elapsed by the preset time t, the CPU 34 returns to step S40A, and then obtains the coordinates of the illuminated point, as described above. Accordingly, coordinates of a plurality of illuminated points are memorized until the time (measured by the timer 33) elapses by the preset time t. When the time measured by the timer 33 has elapsed by the preset time t, the CPU 34 proceeds to step S60A.

### (Step S60A)

In step S60A, the CPU 34 averages the respective coordinates of the illuminated points, in the illuminated point detecting section 70, obtained until the time elapses by the preset time t, to compute the coordinates of the average of the illuminated points. The coordinates of the average are memorized in the memory section 32, correspondingly to the value k of the counter.

When the value k of the counter is 0, the resultant coordinates of the average are memorized in the memory section 32 as the coordinates of the average of the illuminated points when the value k of the counter is 0. The coordinates of the average of the illuminated points when the value k of the counter is 0 are referred to as coordinates of a first average. When the value k of the counter is 1, the resultant coordinates of the average are memorized in the memory section 32 as the coordinates of the average of the illuminated points when the value k of the counter is 1. The coordinates of the average of the illuminated points when the value k of the counter is 1 are referred to as coordinates of a second average. When the CPU 34 has obtained the coordinates of the average of the illuminated points, in the same way as in the first embodiment, the CPU 34 causes the output section 50, for example, the display screen of the display, to display a symbol showing the position of the coordinates of the average.

### (Step S90A)

The coordinates of the center C of the irradiation plane 80a are memorized as already known data in the memory section 32. In step S90A, the CPU 34 computes the neck-angle difference in the same way as in the first embodiment.

The second embodiment has the following characteristics.

(1) The measuring apparatus of the second embodiment has the radiating section 10 fitted to a subject's head to radiate a spot ray; and the illuminated point detecting section 70 including the irradiation plane 80a to be irradiated with the spot ray radiated from the radiating section 10 and further detects the position of an illuminated point of the spot ray on the irradiation plane 80a.

The illuminated point detecting section 70 detects respective positions of illuminated points, each of which corresponds to the illuminated point, in the first period of time, for which the subject keeps his/her face forward, thereby obtaining a first detection data group of the respective positions of the illuminated points, and detects respective positions of illuminated points, each of which corresponds to the illuminated point, in the second period of time, for which the subject turns his/her neck horizontally from the front and subsequently returns his/her neck or head to keep his/her face forward again, thereby obtaining a second detection data group of the respective positions of the illuminated points. The measuring apparatus further has the computer 30, which computes a difference between the angle of the subject's neck in the first period of time, and that in the second period of time. The computer 30 computes coordinates of the first average of the illuminated points on the irradiation plane 80a in the first detection data group, and coordinates of the second average of the illuminated points on the irradiation plane 80a in the second detection data group; obtains the distance between the point of the coordinates of the first average and a perpendicular line that extends from a point of the coordinates of the second average and orthogonally to a horizontal line drawn to pass onto a point of the coordinates of the first average. The computer 30 computes the neck-angle difference on the basis of the distance between the perpendicular line and the coordinates of the first average, and the distance D1 between the radiating section 10 and the irradiation plane 80a. As a result thereof, according to the measuring apparatus of the second embodiment, the neck-angle difference can be precisely measured without needing to give a mark onto any one of the illuminated points.

(2) The measuring apparatus of the second embodiment includes the trigger input section 40 for inputting a trigger signal into the computer 30. When the trigger signal is input from the trigger input section 40 to the computer 30, the timer 33 is activated to start measuring the first and second periods of time. When the time measured by the timer 33 has elapsed by the preset time t, the first and second periods of time each end. As a result thereof, the apparatus achieves an advantage equivalent to the advantage (2) produced by the first embodiment.

The above-mentioned embodiments may be modified as follows.

In the first embodiment, the image pickup section 20 is configured to pick up a moving image. However, the image pickup section 20 may be configured to pick up a plurality of still images continuously. In this case, it only necessary to convert the still images continued intermittently to digital images, and then compute the coordinates of any illuminated point on the basis of the digital images.

In the first embodiment, the screen 60 may be changed into a wall of a building.

In the first and second embodiments, whenever the preset time t, for which the subject keeps his/her face forward in each of the first and second neck-angle measurements, elapses, the coordinates of the average are computed in each of steps S60 and S60A. Instead of this manner, it is allowable to: memorize, when coordinates of an illuminated point are obtained, the coordinates into the memory section 32 in step S40 or step S40A in each of the first and second neck-angle measurements; and compute, after a determination of “YES” is made in step S80, coordinates of the first average and coordinates of the second average, respectively, about illuminated points obtained in the first and second neck-angle measurements before step S90 or S90A.

The computer 30 of each of the first and second embodiments may store, in the memory section 32, standard data on the respective neck-angle differences of able-bodied persons as a database. In step S90 of the flowchart in FIG. 2, or step S90A of that in FIG. 8, the computer 30 may compare the standard data on the respective neck-angle differences of the able-bodied persons, for example, a reference range (−γ1<0°<+γ2) of the persons' neck angles with the neck-angle difference e of the subject, which has been computed in step S90 or S90A, to output a result of the comparison into the output section 50. The values γ1 and γ2 are positive values, and are values obtained through tests or the like. In this case, the result of the comparison with the able-bodied persons is obtained. Thus, on the basis of the comparison of the subject with the able-bodied persons, the result can be helpful to a determination as to whether or not the subject has a disorder in the vestibule of his/her semicircular canal, or about the position sensation of his/her neck.

The first and second embodiments have each been applied to an apparatus for measuring a subject's neck-angle difference in a case where the subject turns his/her neck horizontally. However, the present invention may be applied to an apparatus for measuring a subject's neck-angle difference in a case where the subject moves his/her head vertically (or nods his/her head). In this case, the structure of each of the first and second embodiments is kept as it is, and step S90 of the flowchart in FIG. 2 or step S90A of that in FIG. 8 is changed as follows.

In the calculation of the neck-angle difference in step S90 or step S90A, instead of the horizontal line H, for example, the following is used as illustrated in FIG. 6: a vertical line V3 drawn to pass onto the center C (base point) of the irradiation plane 60a and further contained in the irradiation plane 60a.

The CPU 34 uses, as first and second perpendicular lines H1 and H2, a pair of perpendicular lines, which extend from illuminated points P1 and P2 to the vertical line V3, and are perpendicular to the vertical line V3, to compute coordinates of respective intersection points P5 and P6 of the vertical line V3 and the first and second perpendicular lines H1 and H2. Next, the CPU 34 uses the respective coordinates of the intersection points P5 and P6, and the coordinates of the center C (base point) of the irradiation plane 80a to compute the distance from the center C (base point) to the intersection point P5, that is, the distance from the center C (base point) to the first perpendicular line H1, as well as the distance from the center C (base point) to the intersection point P6, that is, the distance from the center C (base point) to the second perpendicular line H2. On the basis of these distances, and the distance from the radiating section 10 to the center C (base point), the neck-angle difference is computed in the same way as in the first embodiment. The illuminated point P1 in this modification is an illuminated point when the subject keeps his/her face forward before the subject turns his/her neck. The second illuminated point P2 is an illuminated point when the subject returns his/her face to the front after the subject moves his/her head upward or downward.

In each of the embodiments, the trigger input section 40 is provided. However, the trigger input section 40 may be omitted to change the structure of the embodiment as follows. A time when the first period of time is started and a time when the first period of time is ended may be set up through the timer 33. In this case, the measuring apparatus informs the subject of the start and the end of the first period of time on the basis of time-measuring by the timer 33. During the first period of time, images are picked up through the image pickup section 20. Thereafter, the measuring apparatus instructs the subject to turn his/her neck or move his/her head upward or downward. Next, the measuring apparatus informs the subject of the start and the end of the second period of time, for which the subject turns his/her neck to direct his/her face to the front. During the second period of time, images are picked up through the image pickup section 20.

In this case, the computer 30 automatically informs the subject of the start and the end of each of the periods of time, and the above-mentioned instruction.

In each of the first and second embodiments, the first and second periods of time are the same preset time t (for example, a period of 100 to 10000 ms). However, the first and second periods of time may be different from each other.

### First Modification of First Embodiment

According to the flowchart of the first embodiment, after the coordinates of the first average are obtained in the first period of time and those of the second average are obtained in the second period of time, the neck-angle difference is computed in step S90 to end the program. However, this manner may be changed as follows.

In the first period of time, the coordinates of the first average are obtained. The obtained coordinates are used as reference coordinates.

In step S80, instead of making the determination as to whether or not the value k of the counter is 2, the CPU 34 makes a determination as to whether or not the value k of the counter is N. The number N is an integer that is greater than or equal to 2 and set through the data input section 35. When N is 2, an attained manner is the same as in the first embodiment. When N is 3 or more, coordinates of the average of illuminated points are obtained until the value k of the counter turns to a value of 3 to 4.

When in this case the coordinates of the average obtained in the first neck-angle measurement are used as the coordinates of the above-mentioned first average, coordinates of the average obtained in each of second, third, . . . N^{th} neck-angle measurements are used as the coordinates of the above-mentioned second average in step S90. On the basis of the reference coordinates (the coordinates of the first average), a difference between the neck coordinates is computed in each of the neck-angle-deference measurements.

In this manner, a plurality of neck-angle differences can be computed.

### Second Modification of First Embodiment

The first modification of the first embodiment may be further modified as follows.

In the first modification of the first embodiment, the coordinates of the average obtained in each of second, third, . . . N^{th} neck-angle measurements are used as the coordinates of the second average. However, this manner may be changed as follows. The coordinates of the average obtained in the first neck-angle measurement are used as the coordinates of the first average, and the coordinates of the average obtained in the second measurement are used as the coordinates of the second average. Next, the coordinates of the average obtained in the second neck-angle measurement are used as the coordinates of the first average, and the coordinates of the average obtained in the third measurement are used as the coordinates of the second average. In such a way, the coordinates of the average obtained in the (N−1)^{th} neck-angle measurement are used as the coordinates of the first average, and the coordinates of the average obtained in the N^{th} measurement are used as the coordinates of the second average. On the basis of the coordinates of each of the references (the coordinates of each of the first averages), a difference between the neck coordinates is computed.

In this way, a plurality of neck-angle differences can be computed.

The second embodiment may be modified in the same way as in each of the first and second modifications of the first embodiment.

Therefore, the present examples and embodiments are to be considered as illustrative and not restrictive and the invention is not to be limited to the details given herein, but may be modified within the scope and equivalence of the appended claims.

1. An apparatus for measuring a difference between angles of a neck of a subject, the apparatus comprising:
a radiating section fitted to a head of the subject to radiate a spot ray onto an irradiation plane;
an image pickup section, which picks up an image of the irradiation plane that includes an illuminated point of the spot ray radiated from the radiating section, wherein the image pickup section picks up an image of the irradiation plane in a first period of time for which the subject faces forward, thereby obtaining a first image group including a plurality of images, and further picks up an image of the irradiation plane in a second period of time, which is after the neck of the subject either turns horizontally or vertically from the front and subsequently returns the neck of the subject to face forward again, thereby obtaining a second image group comprising a plurality of images; and
a computer, which computes a neck-angle difference, the neck-angle difference defined as a difference between an angle of the neck of the subject in the first period of time and an angle of the neck of the subject in the second period of time, wherein the computer
computes coordinates of a first average of illuminated points, each of which corresponds to the illuminated point, on the irradiation plane in the individual images contained in the first image group, and coordinates of a second average of illuminated points, each of which corresponds to the illuminated point, on the irradiation plane in the individual images contained in the second image group,
obtains a distance between a base point on the irradiation plane and a first perpendicular line, which extends from a point of the coordinates of the first average and orthogonally to a horizontal line or a vertical line that is included in the irradiation plane and further drawn to pass onto the base point,
obtains a distances between the base point and a second perpendicular line, which extends from a point of the coordinates of the second average and orthogonally to the horizontal or the vertical line, and
computes the neck-angle difference on the basis of the distance between the first perpendicular line and the base point, the distance between the second perpendicular line and the base point, and the distance between the radiating section and the base point on the irradiation plane.

2. The apparatus according to claim 1, wherein
the computer includes a trigger input section for inputting a trigger signal and a timer for measuring time,
when the trigger signal is input from the trigger input section to the computer, the timer is activated to start to measure time for each of the first period of time and the second period of time, and
when the time measured by the timer has elapsed by a predetermined time, the time-measuring for each of the first period of time and the second period of time is finished.

3. The apparatus according to claim 1, wherein
an output section for outputting the computed neck-angle difference is connected to the computer, and
the computer compares standard data as a difference between angles of a neck of an able-bodied person with the computed difference between the angles of the neck of the subject and outputs a result from the comparison to the output section.

4. An apparatus for measuring a difference between angles of a neck of a subject, the apparatus comprising:
a radiating section fitted to a head of the subject to radiate a spot ray;
an illuminated point detecting section, which includes an irradiation plane to be irradiated with the spot ray radiated from the radiating section and further detects the position of an illuminated point of the spot ray on the irradiation plane, wherein the illuminated point detecting section detects respective positions of illuminated points, each of which corresponds to the illuminated point, in a first period of time for which the subject faces forward, thereby obtaining a first detection data group of the respective positions of the illuminated points, and detects respective positions of illuminated points, each of which corresponds to the illuminated point, in a second period of time, which is after the neck of the subject either turns horizontally or vertically from the front and subsequently returns the neck of the subject to face forward again, thereby obtaining a second detection data group of the respective positions of the illuminated points; and
a computer, which computes a neck-angle difference, the neck-angle difference defined as a difference between an angle of the neck of the subject in the first period of time and an angle of the neck of the subject in the second period of time, wherein the computer
computes coordinates of a first average of the illuminated points on the irradiation plane in the first detection data group, and coordinates of a second average of the illuminated points on the irradiation plane in the second detection data group,
obtains a distance between the point of the coordinates of the first average and a perpendicular line that extends from a point of the coordinates of the second average and orthogonally to a horizontal line drawn to pass onto a point of the coordinates of the first average, and
computes the neck-angle difference on the basis of the distance between the perpendicular line and the coordinates of the first average, and the distance between the radiating section and the irradiation plane.

5. The apparatus according to claim 4, wherein
the computer includes a trigger input section for inputting a trigger signal and a timer for measuring time,
when the trigger signal is input from the trigger input section to the computer, the timer is activated to start to measure time for each of the first period of time and the second period of time, and
when the time measured by the timer has elapsed by a predetermined time, the time-measuring for each of the first period of time and the second period of time is finished.

6. The apparatus according to claim 5, wherein
an output section for outputting the computed neck-angle difference is connected to the computer, and
the computer compares standard data as a difference between angles of a neck of an able-bodied person with the computed difference between the angles of the neck of the subject and outputs a result from the comparison to the output section.
